(19) United States
(12) Patent Application Publication
(10) Pub. No.: US 2024/0165127 A1
(43) Pub. Date: May 23, 2024

(54) CONTRAST AGENT FOR DIAGNOSIS OF VASCULAR DISEASE CONTAINING GADOLINIUM-BASED COMPOUND AS ACTIVE INGREDIENT AND SYNTHESIS METHOD THEREFOR

(71) Applicant: DAEGU CATHOLIC UNIVERSITY INDUSTRY ACADEMIC COOPERATION FOUNDATION, Gyeongsan-si (KR)

(72) Inventors: Sung Won YOUN, Daegu (KR); Phuong Tu HUYNH, Daegu (KR); Huy Duc VU, Daegu (KR); Jung-hwa RYU, Daegu (KR)

(73) Assignee: DAEGU CATHOLIC UNIVERSITY INDUSTRY ACADEMIC COOPERATION FOUNDATION, Gyeongsan-si (KR)

(21) Appl. No.: 18/496,519

(22) Filed: Oct. 27, 2023

(30) Foreign Application Priority Data
Nov. 3, 2022 (KR) 10-2022-0145087

(51) Int. Cl. A61K 31/555

(52) U.S. Cl. CPC A61K 31/555; A61K 2123/00

(57) ABSTRACT

There is provided a contrast agent for diagnosis of vascular disease containing a gadolinium-based compound as an active ingredient. It has been found that the gadolinium-based compound Gd-DOTA-click-SF of the present disclosure, has a higher molar longitudinal relaxivity (r₁) than Dotarem, is stable, and does not affect in vitro cell viability. In addition, it has been found through MRI images that the specific binding of Gd-DOTA-click-SF in an animal model of abdominal aortic aneurysm established using elastase, a serine protease, is remarkable. Also, it has been confirmed that the content of the gadolinium-based compound of the present disclosure that binds to serine protease in the aorta, that is, the content of gadolinium in the aorta, is remarkable. Thus, the Gd-DOTA-click-SF compound according to the present disclosure may be usefully used in MRI and CT imaging diagnosis for vascular disease.

### CROSS-REFERENCE TO PRIOR APPLICATION

This application claims priority to Korean Patent Application No. 10-2022-0145087 (filed on Nov. 3, 2022), which is hereby incorporated by reference in its entirety.

### BACKGROUND

The present disclosure relates to a contrast agent for diagnosis of vascular disease containing a gadolinium-based compound as an active ingredient and a synthesis method therefor.

Abdominal aortic aneurysm (AAA) is a degenerative disease involving chronic aortic wall inflammation, degradation and remodeling of structural extracellular matrix (ECM) proteins, and progressive expansion of the aortic wall. Elastase catalyzes the degradation of elastin and is used to establish an abdominal aortic aneurysm model.

Serine proteases constitute the largest family of proteases known to play important roles in many cellular processes, including cell differentiation, blood coagulation, extracellular matrix degradation and remodeling, apoptosis, and inflammation. Thereamong, elastase, which may be used as an inflammation marker, is an enzyme belonging to the serine protease family, consists of 240 amino acids, contains four disulfide bridges, and has a molecular weight of 25.9 KDa.

Protease assays and sensor methods have been developed, such as fluorescence-based techniques, colorimetric assays, electrochemical methods, or enzyme-linked peptide protease assays. These are non-invasive measurement methods, but cannot perform in vivo imaging and provide information about the exact location of the active enzyme within the cell or whole organism. That is, efficient detection and imaging of serine proteases in living animals has not yet been successful.

Meanwhile, magnetic resonance imaging (MRI) is a technology that places a human body in a large magnetic container that generates a magnetic field, and then generates high frequencies to resonate protons in the body and measures the difference in signals from each tissue, and reconstructs and images the signals by means of a computer.

Compared to other imaging methods, MRI has the advantage of having high resolution and contrast and being able to provide images of deep organs and 3D information in real time, and thus is widely used for diagnosis.

MRI can bioimage unique tissue signal differences using various techniques, but when a contrast agent is used, it can further increase the contrast of tissue by changing the self-relaxation time of water molecules in the tissue, thereby increasing visibility, which makes a more accurate diagnosis possible.

Computed tomography (CT), which uses X-rays, is effective for imaging dense structures like bones, but when it is used to image soft tissue, it has limitations in distinguishing the tissue from surrounding areas because the clarity is greatly reduced. In order to overcome these limitations of CT, many studies have been conducted on contrast agents for CT, and currently, iodine-based contrast agents such as Ultravist (trade name, Bayer, USA) are most widely used.

Gadolinium-based contrast agents are used in magnetic resonance imaging (MRI) and tissue computed tomography (CT). Gadolinium itself is a substance toxic to the human body, and thus gadolinium may be chelated with a ligand such as diethylene triamine pentaacetic acid (DTPA), or 1,4,7,10-tetraazacyclododecane-1,4,7,10-tetraacetic acid (DO3A), to counteract the toxicity thereof, and may be used as a Gd-DTPA, Gd-DOTA, or Gd-DO3A complex. Since the toxicity of gadolinium element is due to the fact that gadolinium has many binding sites that can react with biomolecules in vivo, gadolinium is chelated with a ligand to block the binding sites through which gadolinium can react with biomolecules, thereby reducing the toxicity of gadolinium.

This metal complex is required to retain its chemical stability in the human body. As used herein, the term “chemical stability” refers to how long the metal complex retains its stability without being separated. If the Gd-DTPA or the like is separated into gadolinium and the ligand before being excreted out of the human body, it will cause fatal results. Such chemical stability is often expressed as a stability constant, which is a thermodynamic concept. Meanwhile, because a contrast agent with a high self-relaxation rate shows a high-contrast enhancement effect even when administered in a relatively small amount, the high self-relaxation rate in MRI images is a factor that proves the high efficiency of the MRI contrast agent. In general, it is desirable that an MRI contrast agent has a high self-relaxation rate and is thermodynamically stable.

As technology related to gadolinium complexes as MRI and CT contrast agents, Korean Patent No. 2386595 discloses a novel gadolinium-based compound, a method for producing the same, and an MRI contrast agent containing the same, and Korean Patent No. 1668189 discloses a gadolinium complex, an MRI-CT dual imaging contrast agent containing the same, and a method for producing the gadolinium complex. However, so far, a contrast agent for diagnosis of vascular disease containing a gadolinium-based compound as an active ingredient and a method for synthesizing the same according to the present disclosure have not been disclosed.

### SUMMARY

The present disclosure has been made to satisfy the above-mentioned needs, and an object of the present disclosure is to provide a contrast agent for diagnosis of vascular disease containing a gadolinium-based compound as an active ingredient and a method for synthesizing the same. The inventors of the present disclosure have found that Gd-DOTA-click-SF, a gadolinium-based compound, has a higher molar longitudinal relaxivity (r₁) than Dotarem, is stable, and does not affect in vitro cell viability, and have found through MRI images that the specific binding of Gd-DOTA-click-SF in an animal model of abdominal aortic aneurysm established elastase, a serine protease, is remarkable, and have confirmed that the content of the gadolinium-based compound of the present disclosure that binds to serine protease in the aorta, that is, the content of gadolinium in the aorta, is remarkable, thereby completing the present disclosure.

To achieve the above object, the present disclosure provides a contrast agent for diagnosis of vascular disease containing a Gd-DOTA-click-SF compound of Formula 1 as an active ingredient.

The present disclosure also provides a method for synthesizing a Gd-DOTA-click-SF compound including steps of:
(1) synthesizing Gd-azido-DOTA by allowing azide-mono-amide-DOTA to react with GdCl₃·6H₂O; and
(2) synthesizing Gd-DOTA-click-SF by subjecting the Gd-azido-DOTA synthesized in step (1) to click reaction with SF-alkyne.

The present disclosure also provides a Gd-DOTA-click-SF compound of Formula 1.

The present disclosure provides a contrast agent for diagnosis of vascular disease containing a gadolinium-based compound as an active ingredient and a method for synthesizing the same. It has been found that Gd-DOTA-click-SF, a gadolinium-based compound, has a higher molar longitudinal relaxivity (r₁) than Dotarem, is stable, and does not affect in vitro cell viability. In addition, it has been found through MRI images that the specific binding of Gd-DOTA-click-SF in an animal model of abdominal aortic aneurysm established using elastase, a serine protease, is remarkable. In addition, it has been confirmed that the content of the gadolinium-based compound of the present disclosure that binds to serine protease in the aorta, that is, the content of gadolinium in the aorta, is remarkable.

### BRIEF DESCRIPTION OF THE DRAWINGS

FIG. 1 shows processes of synthesizing a Gd-azido-DOTA complex (A) and a Gd-DOTA-click-SF probe (B).

FIG. 2 shows the results of analyzing whether Gd-azido-DOTA was synthesized. Specifically, FIG. 2, A shows the results of thin layer chromatography (TLC) performed to follow the reaction of Gd-azido-DOTA (N₃: azido-DOTA spot, Co: combined spots of azido-DOTA and reaction mass, and RM: reaction mass spot), and FIG. 2, B shows the HR-FAB mass spectrum of Gd-azido-DOTA.

FIG. 3 shows the results of analyzing whether Gd-DOTA-click-SF was synthesized. Specifically, FIG. 3, A shows the results of thin layer chromatography (TLC) of Gd-DOTA-click-SF after purification (SF-Alk: SF-alkyne, Gd-N₃: Gd-azido-DOTA, and P: product Gd-DOTA-click-SF after purification), and FIG. 3, B shows the HR-FAB mass spectrum of Gd-DOTA-click-SF.

FIG. 4 shows the results of examining the relaxivity (r₁) of gadolinium (Gd) at various concentrations (0, 0.01, 0.02, 0.04, 0.06, 0.08 and 0.1 mM). Specifically, FIGS. 4, A and B show the slopes of the molar longitudinal relaxivities (R₁) of Dotarem and Gd-DOTA-click-SF, respectively, determined using the inversion recovery method, and FIG. 4, C shows the MRI phantoms of Dotarem and Gd-DOTA-click-SF.

FIG. 5, A shows an MRI phantom plan, FIG. 5, B shows the region of interest (ROI) of T1 mapping on MRI, and FIG. 5, C shows the relaxivity (R₁) of gadolinium at various concentrations (0.01, 0.02, 0.04, 0.06, 0.08 and 0.1 mM).

FIG. 6, A shows an MRI phantom plan, FIG. 6, B shows the region of interest (ROI) of T1 mapping on MRI, and FIG. 6, C shows the relaxivity (R₁) of gadolinium at various concentrations (0.04, 0.08, 0.16, 0.32 and 0.64 mM).

FIG. 7 shows the Gd(III) content (%) in each of Gd-DOTA-click-SF (Gd-SF) and Dotarem in PBS buffer solution and ZnCl₂ (250 mM) solution as a function of incubation time.

FIG. 8 shows the results of CCK-8 assay performed to analyze the cell viability of the RAW 264.7 cell line (A) and the C166 endothelial cell line (B) in the presence of each of Gd-DOTA-click-SF and Dotarem.

FIG. 9 shows the results of MALDI-TOF/TOF mass spectrometry (linear mode) of Gd-DOTA-click-SF that reacted with elastase (MW: 26,819.5078).

FIG. 10 shows the results of MALDI-TOF/TOF mass spectrometry (reflector mode) of Gd-DOTA-click-SF that reacted with somatostatin (MW: 1,637.88).

FIG. 11 shows the results of MALDI-TOF/TOF mass spectrometry (reflector mode) of Gd-DOTA-click-SF that reacted with oxytocin (MW: 1,007.19).

FIG. 12 shows the results of MALDI-TOF/TOF mass spectrometry (reflector mode) of Gd-DOTA-click-SF that reacted with ribonuclease A (MW: 13,683.30).

FIG. 13 shows the results of T1-weighted MRI, obtained by administering each of Dotarem and Gd-DOTA click-SF of the present disclosure as a contrast agent to a normal aorta group and an abdominal aortic aneurysm group (A and D: Lot 1; B and E: Lot 2; and C and F: Lot 3) (\textcircled{1} H-Do, \textcircled{2}: H-Gd-SF, \textcircled{3}: AAA-Do, and \textcircled{4}: AAA-Gd-SF) (D, E, and F:

brightness/contrast of the images shown in A to C).

FIG. 14 shows quantification of the results of T1-weighted MRI. Specifically, FIG. 14, A shows a contrast-to-noise ratio (CNR), and FIG. 14, B is the result of measuring the gadolinium (Gd) content. *** and **** indicate that there are statistically significant differences in CNR or gadolinium content between the compared groups. *** is p<0.001, and **** is p<0.0001.

### DETAILED DESCRIPTION

The present disclosure is directed to a contrast agent for diagnosis of vascular disease containing a Gd-DOTA-click-SF compound of the following Formula 1 as an active ingredient.

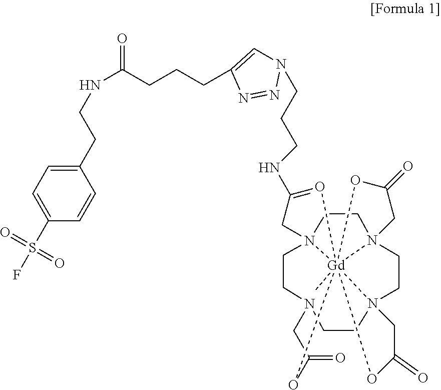

The vascular diseases is preferably any one selected from among congenital vascular disease, myocardial infarction, angina pectoris, stroke, diabetic foot ulcer, intracerebral hemorrhage, cerebral infarction, heart failure, cerebral aneurysm, peripheral vascular disease, aortic dissection, aortic aneurysm, aortic stenosis, varicose vein, venous thrombosis, and vascular inflammation, without being limited thereto.

The contrast agent may preferably be used as a computed tomography (CT) or magnetic resonance imaging (MRI) contrast agent, without being limited thereto. The contrast agent is characterized by having a function of contrasting blood vessel walls.

In addition, the present disclosure is directed to a method for synthesizing a Gd-DOTA-click-SF compound including steps of:
(1) synthesizing Gd-azido-DOTA by allowing azide-mono-amide-DOTA to react with GdCl₃·6H₂O; and
(2) synthesizing Gd-DOTA-click-SF by subjecting the Gd-azido-DOTA synthesized in step (1) to click reaction with SF-alkyne.

The reaction in step (1) is preferably performed by adjusting the pH to 5 to 6 with a 0.05 to 0.15 M KOH solution, and then synthesizing Gd-azido-DOTA by stirring at 50 to 70° C. for 12 to 36 hours, and the reaction in step (2) is preferably performed by synthesizing Gd-DOTA-click-SF by stirring in 80 to 120 mM Tris-HCl (pH 7 to 8), which contains 4 to 6 mM sodium ascorbate, 0.5 to 2.0 mM CuSO₄, and 0.05 to 2.0 mM tributyltin acetate (TBTA), and 15 to 25% (v/v) tert-butanol at room temperature for 12 to 36 hours.

More preferably, the reaction in step (1) is performed by adjusting the pH to 5.5 with a 0.1 M KOH solution, and then synthesizing Gd-azido-DOTA by stirring at 60° C. for 24 hours, and the reaction in step (2) is performed by synthesizing Gd-DOTA-click-SF by stirring in 100 mM Tris-HCl (pH 7.5), which contains 5 mM sodium ascorbate, 1.0 mM CuSO₄, and 0.1 mM tributyltin acetate (TBTA), and 20% (v/v) tert-butanol at room temperature for 24 hours, without being limited thereto.

In addition, the present disclosure is directed to a Gd-DOTA-click-SF compound of the following Formula 1:

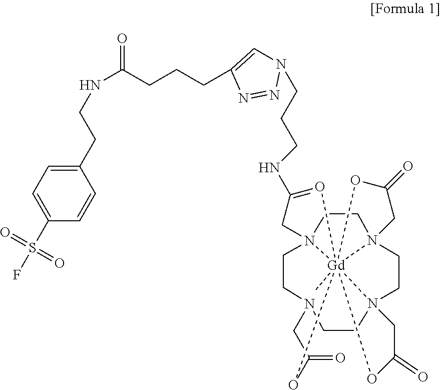

Hereinafter, the present disclosure will be described in more detail with reference to examples. These examples are only for illustrating the present disclosure in more detail, and it will be obvious to those skilled in the art that the scope of the present disclosure is not limited to these examples.

### Example 1. Synthesis of Gd-DOTA-Click-SF Probe

Step 1: Synthesis of Gd-azido-DOTA

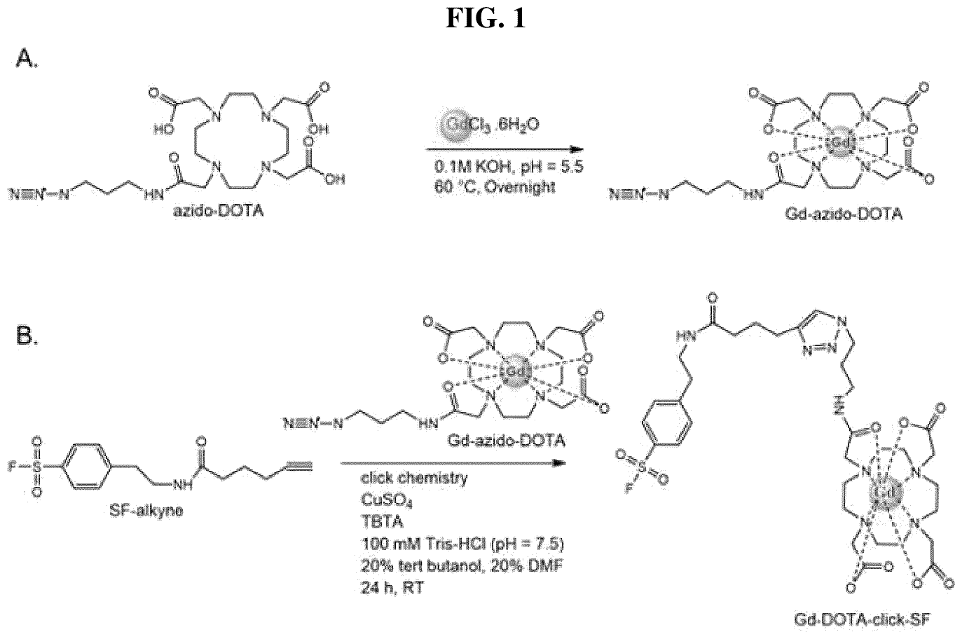

GdCl₃·6H₂O (30.8 mg, 1.3 eq.) was added to a solution of azide-mono-amide-DOTA (38.0 mg, 0.064 mmol, 1 eq.) in 500 μL of deionized water (DI H₂O), and the pH was adjusted to 5.5 with a 0.1 M KOH solution. Then, the mixture was stirred for 24 hours at a temperature of 60° C. to induce the formation of a gadolinium complex (Gd-azido-DOTA) (FIG. 1, A).

The formation of the gadolinium complex was confirmed by thin layer chromatography (TLC) on a C18 silica plate. The plate was developed using a mobile phase (MeOH: 10% CH₃COONH₄ (1:2)).

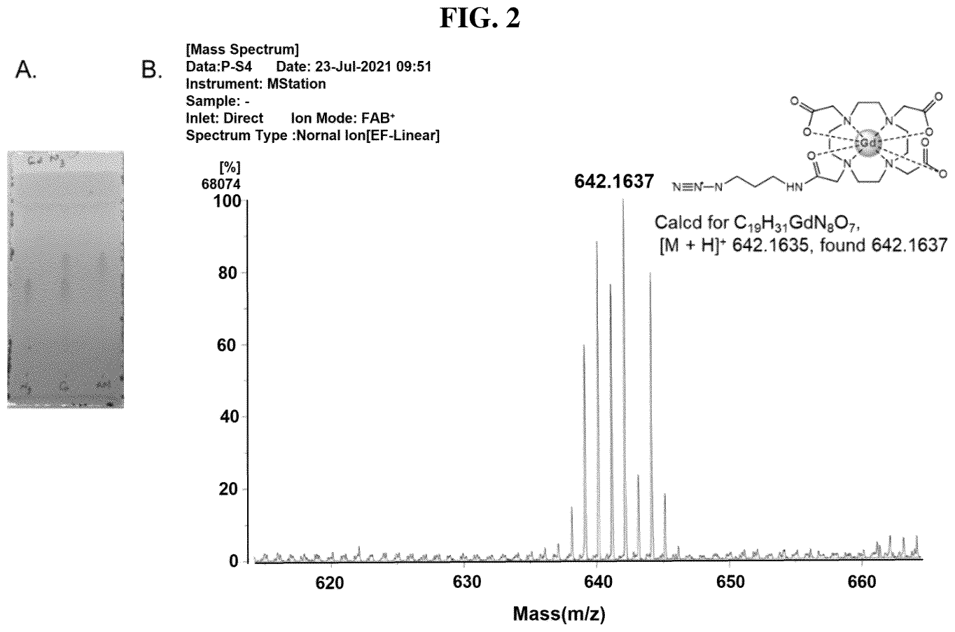

As a result, as shown in FIG. 2, A, it was confirmed in C₁₈ TLC that the Gd-azido-DOTA product spot moved slightly higher than the azide-DOTA spot. This result suggests that the Gd-azido-DOTA product was less adsorbed on the hydrophobic C18 stationary phase because the polarity of the Gd-azido-DOTA product was greater than that of azide-DOTA. Meanwhile, the molecular weight of the Gd-azido-DOTA complex was determined by HR-FAB mass spectrometry, and as a result, the mass calculated for C₁₉H₃₁GdN₈O₇ [M+H]⁺ was 642.1635 and the mass found was 642.1637 (FIG. 2, B).

Thereafter, the mixture was concentrated using a rotary evaporator and then dissolved again in DI H₂O. Free Gd³⁺ ions were removed by adding Chelex 100 resin to the Gd-azido-DOTA solution, and the solution was gently stirred for 2 hours. Next, the supernatant was taken and concentrated using a rotary evaporator.

(2) Step 2: Synthesis of Gd-DOTA-Click-SF Probe from Gd-Azido-DOTA and SF-Alkyne (FIG. 1, B)

The click chemical reaction between Gd-azido-DOTA (42 mg, 0.065 mmol, 1.1 eq.) and SF-alkyne (18 mg, 0.06 mmol, 1 eq.) was performed in 100 mM Tris-HCl (pH 7.5), 20% tert-butanol and 20% dimethylformamide (DMF) in the presence of sodium ascorbate (57.2 mg, 4.8 eq.), CuSO₄·5H₂O (15 mg, 1 eq.) and tris[(1-benzyl-1H-1,2,3-triazol-4-yl)methyl]amine (TBTA) (32 mg, 1 eq.) (FIG. 1B).

The resulting reaction mixture was stirred at room temperature for 24 hours. The formation of Gd-DOTA-click-SF was confirmed by thin layer chromatography (TLC) on a C18 silica plate. The plate was developed with a mobile phase (MeOH: 10% CH₃COONH₄ (1:1)), and the spots were visualized by iodine staining. Then, the mixture was concentrated on a rotary evaporator and dissolved in DI H₂O, and the crude compound was purified by Sep-Pak Plus Short C₁₈.

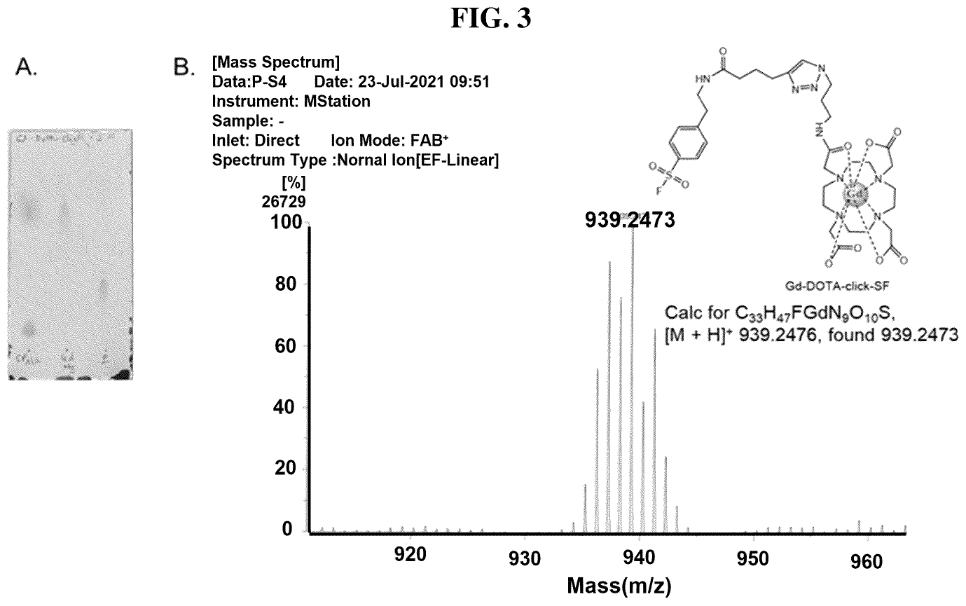

As a result of C₁₈ TLC analysis, it was confirmed that the product of the reaction was more polar than pure SF-alkyne and less polar than Gd-azido-DOTA, and thus appeared in the middle between the SF-alkyne and Gd-azido-DOTA positions (FIG. 3, A). In addition, the molecular weight of the final product was determined by HR-FAB mass spectrometry, and as a result, the mass calculated for C₃₃H₄₇FGdN₉O₁₀S [M+H]⁺ was 939.2476 and the mass found was 939.2473 (FIG. 3, B).

The two-step reaction method for synthesis of the Gd-DOTA-click-SF probe according to the present disclosure finally yielded 29.8 mg of Gd-DOTA-click-SF.

### Example 2. Analysis of Relaxivity of Gd-DOTA-Click-SF Probe

To obtain quantitative information on the efficacy of the Gd-DOTA-click-SF probe and Dotarem as MRI contrast agents, the molar longitudinal relaxivities (r₁) thereof were analyzed. At 25° C., 3.0T MRI was performed using the inversion recovery method, and 9.4T MRI was performed using the RARE (Rapid Acquisition with Refocused Echoes) method.

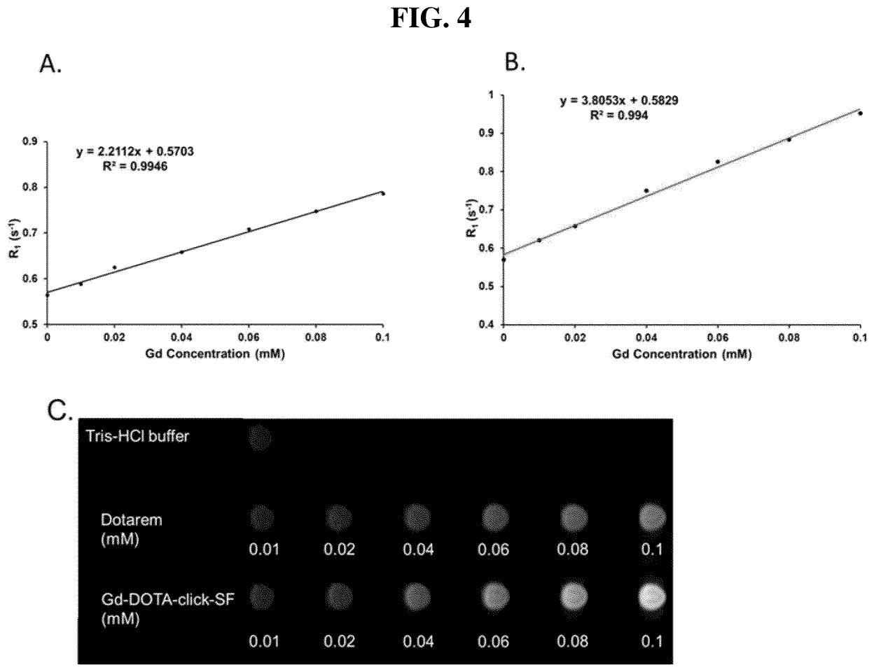

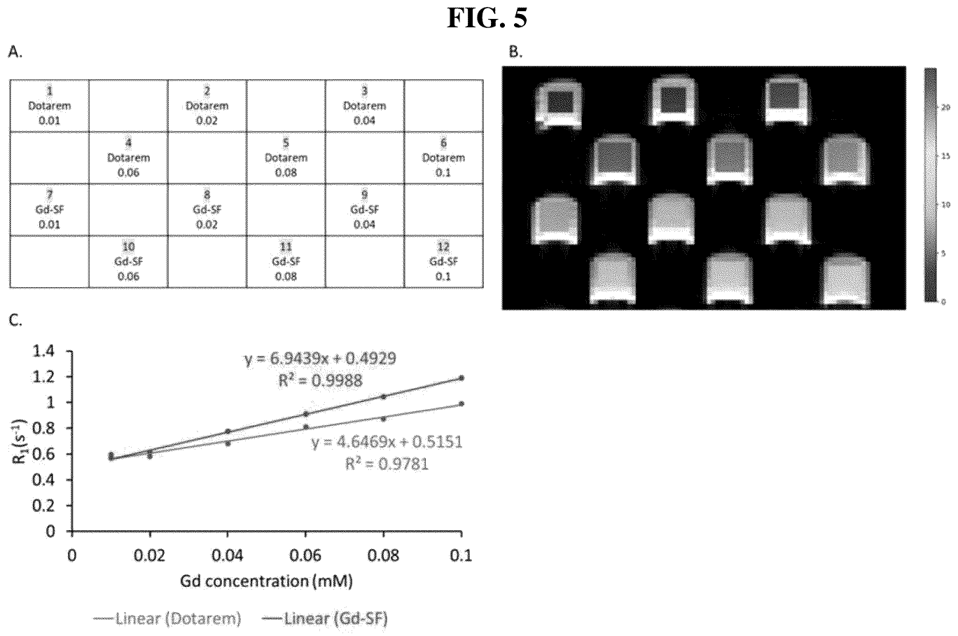

As a result, it was shown that, due to the increased molecular weight of the Gd-DOTA-click-SF probe, the molar longitudinal relaxivity (r₁) of the Gd-DOTA-click-SF probe (r₁=3.805 mM⁻¹s⁻¹) was higher than that of Dotarem (r₁=2.105 mM⁻¹s⁻¹), and the signal intensity of the Gd-DOTA-click-SF probe was higher than that of Dotarem (FIG. 4). These results were consistent with the results obtained in the 9.4T MRI system. In the 9.4T MRI system, the molar longitudinal relaxivity (r₁) of the Gd-DOTA-click-SF probe (r₁=6.944 mM⁻¹s⁻¹) was higher than that of Dotarem (r₁=4.647 mM⁻¹s⁻¹) in the concentration range of 0.01 to 0.1 M (FIG. 5).

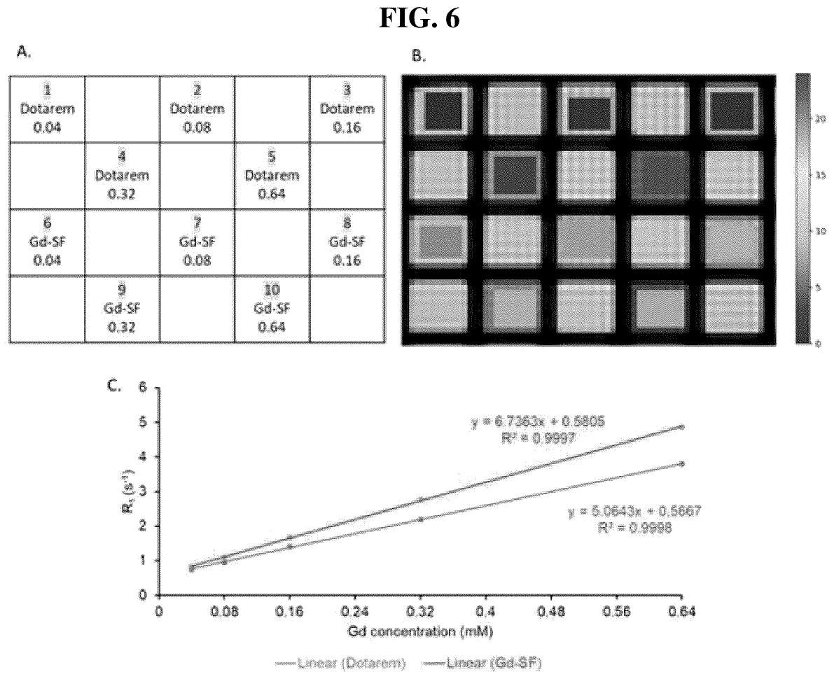

In the concentration range of 0.04 to 0.64 mM, the molar longitudinal relaxivity (r₁) of the Gd-DOTA-click-SF probe (r₁=6.736 mM⁻¹s⁻¹) was shown to be higher than that of Dotarem (r₁=5.064 mM⁻¹s⁻¹) (FIG. 6).

TABLE 1
In vitro relaxivities of Gd-DOTA-click-SF
and Dotarem in 3.0T MRI and 9.4T MRI

| | Molecular | 3.0T r₁ | 9.4T r₁ | |
|---|---|---|---|---|
| Contrast agent | weight (Da) | (mM⁻¹s⁻¹) | (mM⁻¹s⁻¹) | |
| Dotarem | 735.9 | 2.211^{a} | 4.647^{a} | 5.064^{b} |
| Gd-DOTA click-SF | 939.2 | 3.805^{a} | 6.944^{a} | 6.736^{b} |

^{a}Concentrations of Dotarem and Gd-DOTA-click-SF (Gd-SF): 0.01, 0.02, 0.04, 0.06, 0.08, and 0.1 mM
^{b}Concentrations of Dotarem and Gd-DOTA-click-SF (Gd-SF): 0.04, 0.08, 0.16, 0.32, and 0.64 mM

### Example 3. Transmetallation Kinetics

Gadolinium ions are highly toxic in their free form because they do not dissolve at physiological pH, and most of them accumulate in the bones, kidneys, and liver, resulting in very slow systemic excretion. In vivo, the transmetallation of the Gd complex occurs because Gd³⁺ ions compete with other endogenous cations and endogenous anions that destabilize the gadolinium complex and shift the dissociation equilibrium toward its free components. For this reason, the in vitro stability of the Gd(III) complex should be investigated in the presence of Zn(II) ions. 8 μL of a solution of 250 mM ZnCl₂ in phosphate buffer was added to 1.5 mL of 1 mM Gd-DOTA-click-SF complex and the mixture was stirred. Thereafter, the solution was collected at 0, 2, 4, 6, 8, 24, and 48 hours and filtered. The concentration of Gd-DOTA-click-SF was measured by ICP/MS, and the percentage of bound Gd(III) after incubation relative to the value before incubation demonstrated the kinetic inertness of Gd-DOTA-click-SF. To compare the stability of Gd-DOTA-click-SF with that the clinical contrast agent Dotarem, the same procedure was repeated three times.

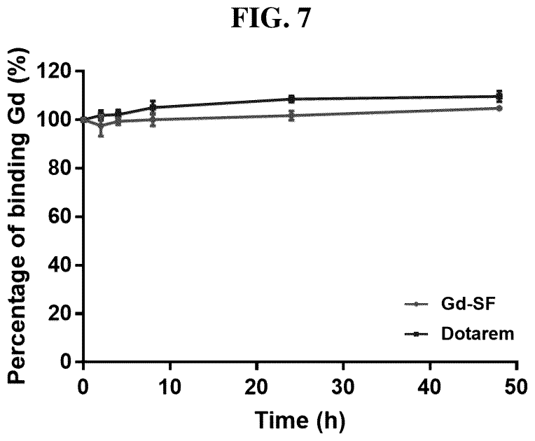

As a result, it was confirmed that the stability of Gd-DOTA-click-SF was comparable to that of Dotarem. Both complexes were shown to be very stable in the presence of Zn(II) ions without transmetallation (FIG. 7).

### Example 4. In Vitro Cell Viability Assay

The cytotoxicity of the Gd-DOTA click-SF probe was assessed against macrophage RAW264.7 cells and endothelial C166 cells using the CCK-8 assay according to the instructions provided by the manufacturer. RAW264.7 cells and endothelial C166 cells were cultured in DMEM containing 10% FBS and 1% penicillin-streptomycin (10000 U/ml). 2.5×10⁴ RAW 264.7 cells and 3×10³ C166 cells per well in 100 μL of DMEM well were seeded in 96-well plates. The cells were cultured at 37° C. for 24 hours in a humidified atmosphere containing 5% CO₂, and then for comparison, each of the Gd-DOTA-click-SF probe and Dotarem was added thereto at concentrations of 0.01, 0.025, 0.05, 0.075, and 0.1 mM, followed by incubation at 37° C. for 24 hours. Thereafter, the cells were treated with 10 μl of CCK-8 solution and further cultured for 2 hours at 37° C., and the absorbance of each well was measured at 450 nm using a microplate reader.

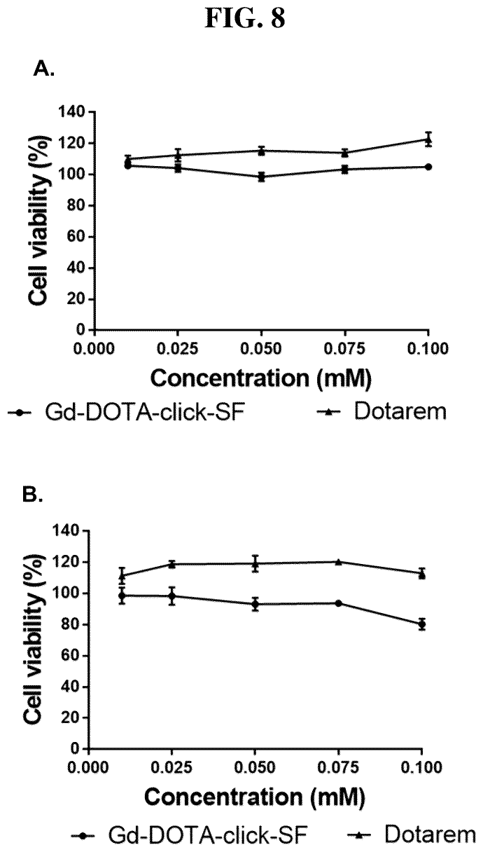

As a result, it was confirmed that the cell viability did not significantly change following treatment with each of Dotarem and the Gd-DOTA-click-SF of the present disclosure, suggesting that both Dotarem and Gd-DOTA-click-SF were non-cytotoxic (FIG. 8).

### Example 6. MALDI-TOF/TOF Mass Spectrometry of Gd-DOTA Click-SF Probe Bound to Serine Protease

To examine the specific targeting of serine proteases, the reaction between Gd-DOTA-click-SF (160 μM) and elastase (belonging to the serine protease family) was investigated.

Additionally, as a comparative example, the reactions between Gd-DOTA-click-SF (160 μM) and other types of peptides (oxytocin, and somatostatin), or and ribonuclease A were analyzed.

Before MALDI-TOF mass spectrometry, the samples were incubated in 100 mM Tris-HCl (pH=7.5) at 37° C. for 1 hour. Detailed instrument setting conditions and mass calibration for MALDI-TOF mass spectrometry are listed in Table 2 below.

TABLE 2
Instrument setting conditions

| | |
|---|---|
| Instrument | MALDI-TOF/TOF ™ 5800 system (AB SCIEX) |

MS parameters

| | |
|---|---|
| Operating mode | MS Linear mode (positive) |
| Mass range (m/z) | 5 to 50 kDa |
| Matrix (concentration and | Sinapinic acid (SA): 10 mg/ml (0.1% TFA/30% |
| solution) | acetonitrile) |
| MS calibration: mix3 | Insulin (bovine) (charge +1, average 5,734.59) |
| | Thioredoxin (E. coli) (charge +1, average 11,674.48) |
| | Apomyoglobin (horse) (charge +1, average 16,952.56) |
| | Apomyoglobin (horse) (charge +2, average 8,476.78) |
| Sample preparation | Matrix:sample = 29:1 |
| Operating mode | MS reflector mode (positive) |
| Mass range (m/z) | 800 to 4,000 Da |
| Matrix (concentration and | α-Cyano-4-hydroxycinnamic acid: 5 mg/ml (0.1% |
| solution) | TFA/50% ACN) |
| MS calibration: mixture | Arg1-Bradykinin (904.468) |
| | Angiotensin I (1296.685) |
| | Glu1-Fibrinopeptide B (1570.677) |
| | ACTH (1-17) (2,093.087) |
| | ACTH (18-39) (2,465.199) |
| | ACTH (7-38) (3,657.9294) |
| MS Calibration | m/z 0.1 |
| Sample preparation | Matrix:sample = 1:1 |
| Data processing | Baseline correction |
| | Noise filter/smooth (Gaussian smooth 9 points) |
| | Mass calibration |

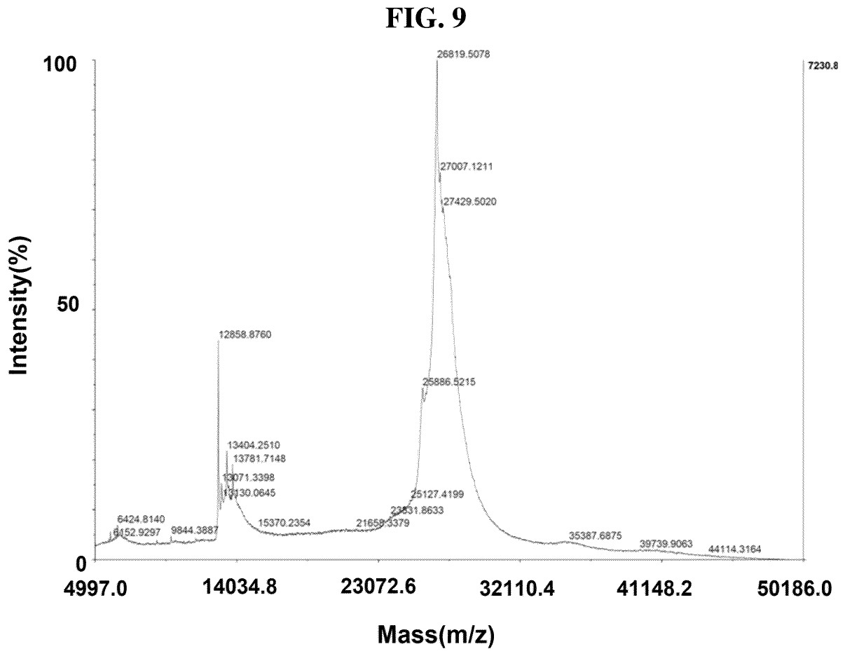

First, as a result of analyzing the click reaction between the Gd-DOTA-click-SF probe and the OH group of the serine residue (elastase), which covalently binds to the Gd-DOTA-click-SF probe, by MALDI-TOF/TOF, it was confirmed that Gd-DOTA-click-elastase was formed and the molecular weight of Gd-DOTA-click-elastase was 26,819.5078 Da, which matches the theoretical molecular weight (26,819.6 Da) (FIG. 9).

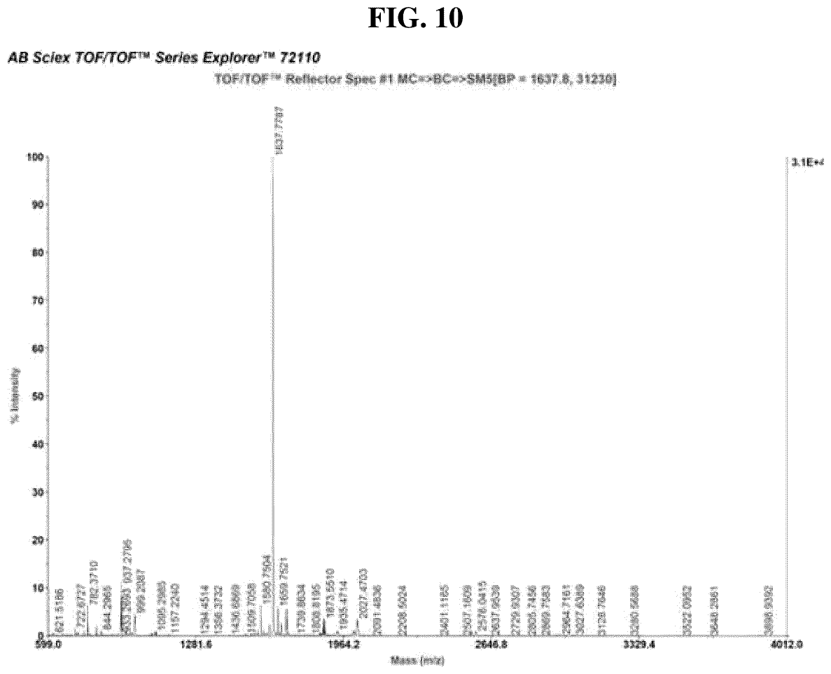

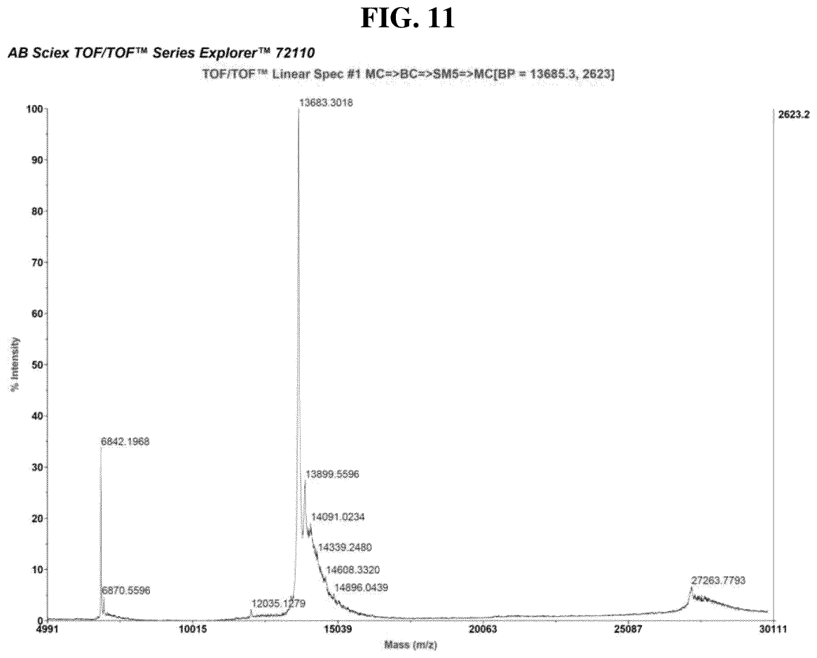

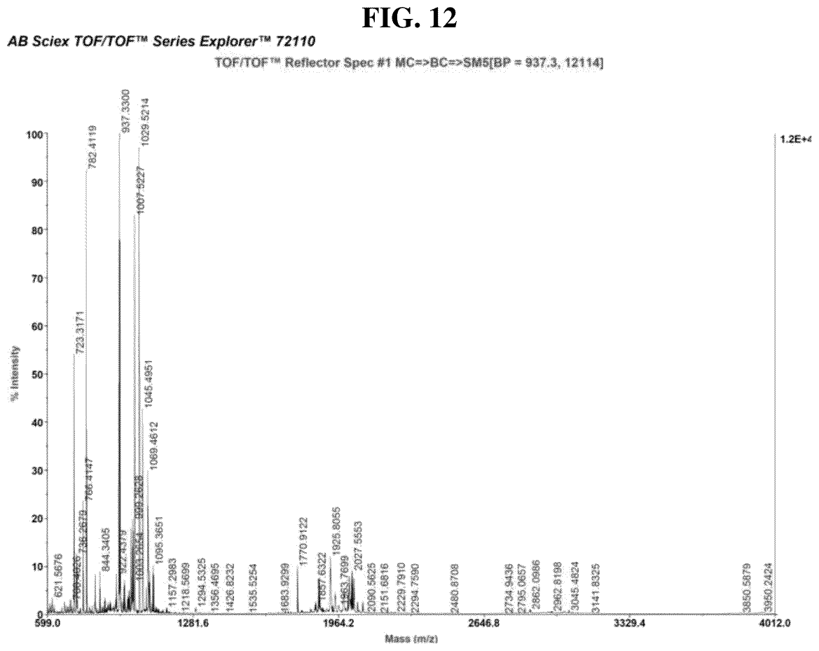

As shown in FIGS. 10 to 12, it was confirmed that the other peptide proteins did not react with Gd-DOTA-click-SF.

### Example 7. Ex Vivo T1-Weighted MRI for Tracking Elastase Activity in Rats with Induced Abdominal Aortic Aneurysm (AAA)

Ex vivo T1-weighted MRI for tracking elastase activity was performed in rats with experimentally induced abdominal aortic aneurysm (AAA).

As experimental animals, 7-week-old Sprague-Dawley (SD) rats were used. The rats were maintained on a 12-hour light-dark cycle with free access to water and feed for one week of acclimatization. Then, the rats were randomly divided into a normal control group and an experimental group (aortic aneurysm group), and each group was further divided into a Dotarem-administered group and a Gd-DOTA-click-SF-administered group. Accordingly, the rats were divided into a total of 4 groups, each consisting of 4 animals.

To induce an abdominal aortic aneurysm, rats were anesthetized by inhalation with 2 to 2.5% isoflurane, and then their abdomen was incised to expose the aortic branches. Next, 300 μL of 12U porcine pancreatic elastase was perfused for 10 minutes.

After 5 weeks, the aorta with induced abdominal aortic aneurysm was harvested, immersed in Dotarem or the Gd-DOTA-click-SF of the present disclosure at 1.28 mM for 1 hour under shaking. Then, it was taken out and washed 3 times with PBS. Finally, the samples were placed in PCR tubes filled with PBS and were examined by T1-weighted MRI. The same procedure was also applied to the aorta from the healthy normal control group, which was immersed in Dotarem or the Gd-DOTA-click-SF of the present disclosure at 1.28 mM for 1 hour under shaking. Then, it was taken out and washed 3 times with PBS. Finally, the samples were placed in PCR tubes filled with PBS and were examined by T1-weighted MRI.

All animal experimental procedures used in the Examples of the present disclosure were approved and conducted in accordance with the guidelines of the Institutional Animal Care and Use Committee of Catholic University of Daegu (approval number: DCIAFCR-180727-09-Y).

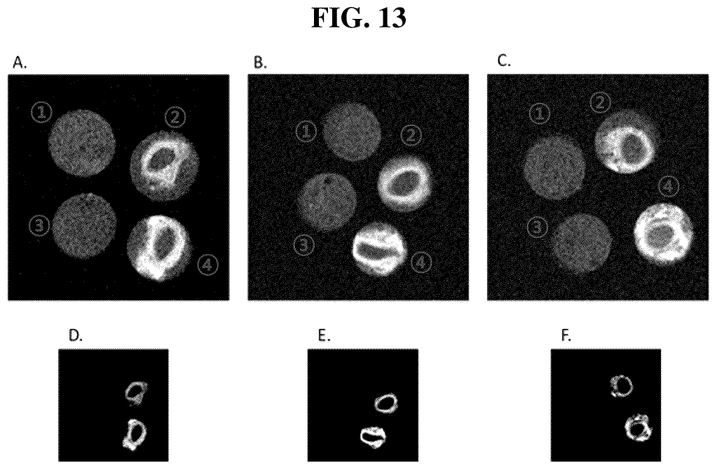

As a result, as shown in FIG. 13, the highest T1-signal intensity was observed in the AAA-Gd-SF samples. The H-Do and AAA-Do samples did not show high signal intensity in T1-weighted MRI, which means that Dotarem was eluted during the washing step and the degree of binding thereof to the arterial wall was very low. H-Gd-SF showed high T1 signal intensity but lower than that of AAA-Gd-SF due to the non-specific binding to the arterial wall.

Therefore, it was confirmed that the contrast agent Gd-DOTA-Click-SF of the present disclosure selectively and effectively binds to the arterial walls with induced abdominal aortic aneurysm, making diagnosis very easy.

### Example 8. Comparison of Contrast Noise Ratios (CNRs) of T1-Weighted MRT1 and of Gadolinium Concentrations ICP/MS Analysis

The CNRs of the MRI results for the four groups, identified in Example 7, were examined, and the concentrations of gadolinium were measured using ICP/MS (ICP/MS 7700, Agilent, USA).

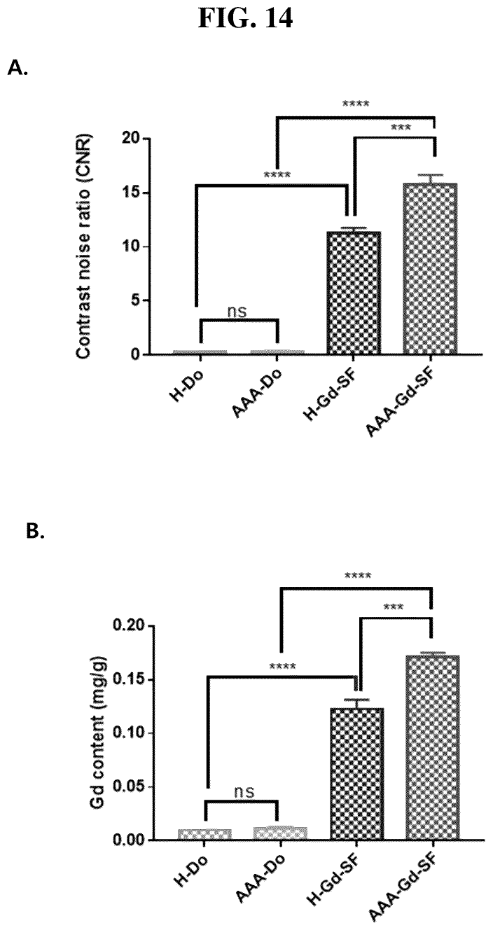

As a result, as shown in FIG. 14, A, the CNR of AAA-Gd-SF significantly increased compared to those of the other groups, and this result was consistent with the ICP/MS results (FIG. 14, B). These results suggest that the contrast agent Gd-DOTA-Click-SF of the present disclosure binds to elastase and enhances the signal intensity in the T1-weighted MRI of the abdominal aortic aneurysm (AAA) group.

What is claimed is:

1. A magnetic resonance imaging (MRI) or computed tomography (CT) contrast agent for diagnosis of vascular disease, the contrast agent containing a Gd-DOTA-click-SF compound of the following Formula 1 as an active ingredient and targeting serine protease in a blood vessel wall:

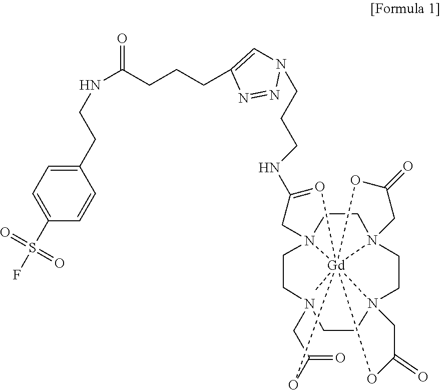

2. The magnetic resonance imaging (MRI) or computed tomography (CT) contrast agent according to claim 1, wherein the vascular diseases is any one selected from among congenital vascular disease, myocardial infarction, angina pectoris, stroke, diabetic foot ulcer, intracerebral hemorrhage, cerebral infarction, heart failure, cerebral aneurysm, peripheral vascular disease, aortic dissection, aortic aneurysm, aortic stenosis, varicose vein, venous thrombosis, and vascular inflammation.
